(19) United States
(12) Patent Application Publication
(10) Pub. No.: US 2006/0005832 A1
(43) Pub. Date: Jan. 12, 2006

(54) Inhaler using pods

(75) Inventors: Alf Niemi, Straengnaes (SE); Sven Calander, Straengnaes (SE); Lars Kax, Nykvarn (SE)

Correspondence Address: OBLON, SPIVAK, MCCLELLAND, MAIER & NEUSTADT, P.C., 1940 DUKE STREET, ALEXANDRIA, VA, 22314, US

(73) Assignee: MEDERIO AG, Hergiswil (CH)

(21) Appl. No.: 11/154,677

(22) Filed: Jun. 17, 2005

(30) Foreign Application Priority Data
Jun. 18, 2004 (SE) SE 0401612-7

(51) Int. Cl. A61M 15/00; A61M 16/00

(52) U.S. Cl. 128/203.15

(57) ABSTRACT

A medicament container for carrying a metered, finely divided, dry powder medication dose is disclosed. The medicament container, which is intended for administration by a dry powder inhaler device, constitutes a pod presents a bottom surface acting as a substrate adapted for receiving a metered medication dose. The metered dose, carried by the pod, is protected from foreign matter, particularly moisture, after a sealing of the pod with a high barrier seal foil, which transforms the pod into a high barrier container. Furthermore the pod is formed such that the pod sealing foil can be slit open from a point of penetration at a first pod end to a point of exit at a second pod end by a opener set in motion relative to the pod.

### REFERENCE TO PRIOR APPLICATIONS

This application claims priority to Sweden 0401612-7 filed Jun. 18, 2004. U.S. Ser. No. 10/898,372 is incorporated herein in its entirety by reference.

### TECHNICAL FIELD

The present invention relates to a method and a device for using metered dry powder medicament doses, loaded in containers, and particularly to single or multiple containers inserted into a dry powder inhaler (DPI) for delivery of the doses.

### BACKGROUND

The dosing of drugs is carried out in a number of different ways in the medical service today. Within health care there is a rapidly growing interest in the possibility of administering medication drugs as a powder directly to the airways and lungs of a patient by means of an inhaler in order to obtain an effective, quick and user-friendly delivery of such substances. Because the efficacy of inhaled doses often are much higher than e.g. orally administered capsules, the inhalation doses need only be a fraction of the medicament powder mass in an oral capsule. Thus, there is an increasing demand for medicament compositions and filling methods for making small and exact inhalation doses of dry powder with low relative standard deviation (RSD).

Volumetric filling is by far the most common method of producing doses of medication drugs. Normally in a first step a quantity of powder is introduced into a receptacle of specified volume by a mechanical device such as a piston or the receptacle may be filled by mean of gravitation and/or suction force. A plurality of receptacles may be arranged in a dose-forming tool, which is adapted to a mechanism bringing a plurality of containers, e.g. blisters or capsules, in line with the corresponding receptacles so that doses of powder may be discharged into the containers. The dose-forming receptacle tool may be integrated into a filling machine such that the receptacles can be filled and emptied in a more or less continuous, cyclic fashion. Examples of prior art may be studied for instance in publications EP 0 319 131 B1, WO 95/21768, U.S. Pat. No. 5,826,633, U.S. Pat. No. 6,267,155 B1, U.S. Pat. No. 6,581,650 B2, DE 202 09 156 U1, WO 03/026965 A1, WO 03/66436 A1 and WO 03/66437 A1.

The active substance in dry powder form, suitable for inhalation needs to be finely divided or otherwise formulated so that the majority by mass of particles in the powder is between 1 and 5 μm in aerodynamic diameter (AD). Powder particles larger than 5 μm tend not to deposit in the lung, when inhaled but to stick in the mouth and upper airways and where they are medicinally wasted and may even cause adverse side effects.

Regarding drug formulation, there are a number of well-known techniques to obtain a suitable primary particle size distribution to ensure correct lung deposition for a high percentage of the dose. Such techniques include jet-milling, spray-drying and super-critical crystallization. There are also a number of well-known techniques for modifying the forces between the particles and thereby obtaining a powder with suitable adhesive forces. Such methods include modification of the shape and surface properties of the particles, e.g. porous particles and controlled forming of powder pellets, as well as addition of an inert carrier with a larger average particle size (so called ordered mixture).

However, dry powders suitable for inhalation are rarely free flowing but tend to stick to all surfaces they come in contact with and the small particles tend to aggregate into lumps. This is due to van der Waal forces generally being stronger than the force of gravity acting on small particles having diameters of 10 μm or less. Therefore, metering and unloading correct quantities of a dry, inhalable powder composition into a dose container, such as a blister for example, becomes more and more difficult the smaller the nominal dose mass gets. Because most active drugs are very potent, only a fraction of a milligram is needed in a dose in many cases. It is therefore necessary to dilute the drug using a suitable, physiologically inert excipient, e.g. lactose, before manufacturing of doses of the drug commences. Today, nominal inhalation doses of less than 1 mg and even less than 0.5 mg are not unusual. Such small doses are very difficult to meter and fill using prior art methods. See for instance the U.S. Pat. No. 5,865,012 and the PCT publication WO 03/026965 A1.

A common practice in the pharmaceutical industry is to dilute the active substance, in order to increase the nominal dose mass to a level, which a chosen filling method can handle. Typically, volumetric doses in prior art have masses in a range from 5 to 50 mg. This often means that the active substance is diluted by a thousand times or more. It is difficult to ascertain that the mix of active substance and diluent is homogenous and to ensure during dose filling that the amount of active substance in each and every one of the metered doses is correct. If the composition comprises big particles to improve flowability for example, care must be taken in handling the powder in order to avoid particle segregation, which easily happens during transportation and handling of the powder. Big particles tend to stay uppermost and small particles tend to fall to the bottom of a storage cavity, which of course results in inconsistent mixing ratios between the finely divided drug and the big particle excipient in the stored powder.

A more recent prior art method of forming a metered dose utilizes an electrostatic or electro-dynamic field deposition process or combinations thereof for depositing electrically charged particles of a medication powder onto a substrate member, such as an electrostatic chuck or a dosing member. A method of depositing microgram and milligram quantities of dry powders using electric field technology is disclosed in our U.S. Pat. No. 6,592,930 B2, which is hereby incorporated in this document in its entirety as a reference. The method is particularly suitable for forming small doses below 10 mg in mass. An example of a suitable dose of medication powder, formed onto a substrate member, is referred to as an electro-dose. The term electro-dose, presented in our Swedish Patent No. SE 0003082-5 (WO 02/18000), which is hereby incorporated herein by reference, refers to a dose of pre-metered medicament powder intended for use in a dry powder inhaler. The electro-dose is formed from an electro-powder comprising an active powder substance or a dry powder medicament formulation with or without one or more excipients, the electro-dose being formed onto a substrate member, which is part of a dosing member. The so formed electro-dose presents suitable properties in terms of occupied area, powder contour, particle size, mass, porosity, adhesion etc for easy de-aggregation and dispersal into air by the use of a suitable dry powder inhaler device.

Yet another problem facing a user of the described prior art dose manufacturing methods is the problem of de-aggregating the powder composition when the dose is made available in a dry powder inhaler (DPI). Because the first priority in prior art manufacturing is to make doses of an almost free-flowing powder composition in order to achieve consistency between doses and a small variation between powder batches, the ability to de-aggregate the dose in a DPI does not get the same attention. The efficacy of the dose therefore is mediocre; the fine particle fraction of the delivered drug is often less than 25%. A method and a device for aerosolizing and delivering a high fine particle dose to a user inhaling a medicament dose from a dry powder inhaler are disclosed in our publications WO 03/086515 A1 and WO 03/086517 A1, which are hereby incorporated herein by reference.

In U.S. Pat. No. 5,590,645, U.S. Pat. No. 5,860,419, U.S. Pat. No. 5,873,360, U.S. Pat. No. 6,032,666, U.S. Pat. No. 6,378,519 and U.S. Pat. No. 6,536,427 a dry powder inhaler for pre-metered doses in containers, using peelable lid foils, is described and some specific powders intended for inhalation are mentioned. The peelable lid foils are described to be made out of a laminate comprising 50 g/m² bleach kraftpaper, 12 micron polyester (PETP) foil, 20 micron soft temper aluminum foil, 9 g/m² vinylic peelable heat seal lacquer (HSL), sealable to PVC, and a laminated base material comprising 100 micron PVC, 45 micron soft temper aluminum foil and 25 micron oriented polyamide. The HSL is sealed to the PVC layer of the base laminate after the powder is filled into a formed cavity in the base laminate. The above described inhaler opens the powder dose before the inhaler is ready for inhalation and the dose is thereby exposed to the surrounding environment and any possible exhalation moist air from a user. A peelable HSL is typically much more sensitive and difficult to seal than a permanent foil seal and therefore an external high barrier package is normally provided to preserve the inhaler over the shelf-life and have the peelable HSL to protect the powder during the in-use time.

The process of filling is very important since any powder not removed from the heat sealable surfaces will very negatively affect the quality of the seal. Preferred filling methods will not deposit the powder formulation onto the sealing surfaces during the filling process. Examples of machines that use separate machine parts to dose the powder into a pod or cavity or onto a substrate surface are described in WO 03027617 A1, WO 03066437 A1, WO 03066436 A1, WO 03026965 A1, WO 0244669 A1 and DE 100 46 127 A1, DE 202 09 156 U1.

In WO 02/00280 A2 and U.S. Pat. No. 6,655,381 B2, an inhaler comprising a magazine holding a rigid unitary magazine including a plurality of integral reservoirs is described. Each reservoir will hold a pre-metered dose of dry powder sealed with a foil. The foil is described as thin plastic film in WO 02/00280 A2 page 6 line 24, which is inadequate as a high barrier seal.

In WO 03/66470 A1, GB 02 385 020 A, and WO 03/15857 A1 an inhaler using compartments to hold the pharmaceutical formulation is described. The compartments have a first and a second face that will be sealed with a foil. A separate part inside each compartment is designed to rupture the foil before inhalation and the documents discuss weakening special sections in the foil to make the opening easier and more reliable. This weakening of the foil could possibly be a problem, if the dose needs a high barrier seal in order not to deteriorate.

In WO 01/30430 A1 a dosage unit for dry powder medicaments is described. The dosage unit is possible to incorporate into a dry powder inhaler such as the one described in WO 02/00279, the dosage unit having a slidable chamber in a sleeve and an openable closure member possible to fit into the dry powder inhaler device. The dosage unit is described to have a cover of substantially the same diameter as the sleeve or being of a frangible material. A separate part inside the device will then push the cover open or rupture the frangible material.

In U.S. 2002/0033176 A1 a dry powder medicament inhalator is described, which is possible to load with a medicament cartridge. The inhalator uses an inhalation activated flow-diverting means for triggering the delivery of the medicament using a lancet to penetrate the medicament cartridge.

In U.S. 2003/0140923 A1, a way of protecting a container filled with a dry powder is discussed using an “active approach to help if a proper high barrier seal could not be achieved”. In U.S. Pat. No. 6,130,263 and U.S. Pat. No. 5,432,214, a moisture absorbing desiccant is incorporated in the material and formed into cavities and foils to protect a product. These applications and patents discuss the possibility of incorporating a desiccant into the material of the container or into the device or into the outer package for the device. This approach is not new and has been used for more than 20 years on the market by Turbuhaler® from AstraZeneca. Turbuhaler® has inside the device an amount of silica gel or a mixture of different types of desiccants to protect the dry powder during the in-use time and during the shelf-time. Turbuhaler® also has an outer package to protect the device during the time on the shelf before opening. Taifun® from Focus Inhalation is also using a desiccant to protect the dry powder formulation inside the device. The amount of desiccant is normally very small in this type of construction and the demands on the high barrier seal to protect the powder remains the same or else the desiccant may be used up before the product comes into use.

In prior art opening of a container for a metered dose to make the dose accessible for inhalation inside a DPI, is accomplished in many different ways. If dose capsules are used then e.g. the capsule is split in two and the content poured out in an intermediate area in the DPI from where the powder is later aerosolized. Another common method is to punch one or more holes in the capsule, blow air into the capsule and optionally vibrate the capsule such that the powder in the dose can be aerosolized and sucked out of the capsule. In the case of a blister container, the cover foil can be peeled off such that the dose is made available directly from the open blister or else poured out in an intermediate area for inhalation.

A prior art container or capsule is thus opened in a first step and aerosolizing is begun in a second step. The time between step 1 and step 2 is different from one DPI to the next, depending on the deployed technical solution, but in many cases the period is not defined and can be anything up to minutes and hours depending on the actions of the user. This is not acceptable from a medical point of view if the dosage can be detrimentally affected by being exposed to the environment inside or outside of the DPI.

Yet another drawback of prior art containers is that the stream of air sucked in to aerosolize the dose attacks all of the powder in the dose at the same time, so that the shearing power of the air stream is distributed over a large area where the dose is stored and the aggregates and particles in the dose are arbitrarily subjected to very different, uncontrolled shearing forces depending on how the powder and particle clusters in the dose are distributed relative the air stream. Most of the powder in the dose is delivered instantaneously with no control of the timing. Where holes are made in the container, e.g. a capsule or a blister, by a sharp, pointed tool or needle, edges of the broken container material will bend inwards towards the dose and the edges may then disturb the flow of air into the container, such that some parts of the dose are not properly aerosolized and de-aggregated.

In some cases all of the powder in the dose is not subjected to the same power of shearing stress because the airflow is unevenly distributed across or through the dose. This tends to further hamper the delivered fine particle dose and raise the proportion of retained powder in the container.

Another problem is incident to aerosolizing a dose in a prior art container and that is that the speed of the aerosolizing air stream starts at zero when the aerosolization process begins. The consequence is that most of the dose is quickly sucked up in aggregated form and the aggregates cannot then be completely de-aggregated during the transport through the air channel of the DPI before entering the airways of the user.

Because of these drawbacks a high degree of de-aggregation is difficult to achieve consistently, and the delivered fine particle dose is relatively small as a percentage of the metered dose.

To sum up, metered dose inhalers of prior art often leave the powder dose exposed to the surrounding atmosphere for a long time. This depends on the inhaler design and the design of the dose container. Barrier properties of the container embodiments are not discussed, leaving the unanswered question of how adequate protection of the fine particle dose of the enclosed medicament is secured during transportation, storing and in-use. Some prior art products make it necessary to open the container and empty the dose into an aerosolizing chamber before the user can begin an inhalation cycle. In some cases the dose may get exposed to a voluntary or involuntary exhalation from the user before a proper inhalation cycle begins. Sometimes the container is opened by a first action by the user, but the act of inhaling from the opened container is delayed uncontrollably, because the user is somehow distracted. Exposing the powder dose to the atmosphere for any reason, including technical shortcomings of the container-inhaler combination, must be kept as short as possible so that the quality of the dose cannot deteriorate before it is inhaled. Also, there should be no room for behavioural errors on behalf of the user.

Thus, there is a need for improved dry powder medicament doses loaded into new types of high integrity containers adapted for insertion into new, fool-proof inhalers guaranteeing consistent high quality administration of such doses.

### SUMMARY

The present invention discloses a sealed medicament container, a so called pod, for carrying a directly loaded, metered dose of a dry powder medicament, the dose being protected from becoming contaminated by foreign matter, especially moisture, by a sealing foil, whereby the fine particle dose is preserved. The pod is intended for insertion into a dry powder inhaler, e.g. by means of a movable slide, where the sealing foil is being opened and the dose delivered directly from the pod by inhalation while the pod is in motion in or into the inhaler device. A method is also disclosed for delivering a dry powder medicament dose directly from a pod to a user of a DPI, whereby the sealing foil of the pod is opened concurrently with or immediately before aerosolizing and entraining of the powder in the dose into the inhaled air. Optionally the sealing foil and the container constitutes a high barrier container, giving a high level of moisture protection to the enclosed dose.

An objective accomplished by the present invention is that the pod, when made available in a DPI, is not opened until a user starts to inhale through the DPI, optionally such that a set minimum pressure is required from the inhalation effort before an opening operation is released. The sealing foil is thus being opened in the opening operation by a relative motion of an opener element vs. the pod concurrently with an inhalation performed by a user.

Another objective accomplished by the present invention is that the speed of the air stream into the DPI resulting from the inhalation is built up to a high speed before the airstream is directed to the powder dose in the container, the dose made available by the opening operation.

In another aspect of the invention the sealing foil being opened is preferably unfolded away from the dose, whereby a suction nozzle gets free access to all of the powder in the dose during the inhalation.

In still another aspect of the present invention a dry powder inhaler device is disclosed comprising a movable slide and means for moving the slide, where said slide is adapted for receiving at least one replaceable medicament container, pod, carrying an enclosed metered dose, where the pod is to be opened by an opener element in an opening operation to be released after the inhalation has begun. Preferably, a minimum suction must be applied to the inhaler device before the opening operation may be released. The powder of the enclosed dose is therefore sucked up by a suction nozzle unless a minimum speed of the airflow into the nozzle has been established.

The present device is set forth by the independent claim 1 and 21 and the dependent claims 2 to 15 and 22 to 26 respectively, and a method of delivery is set forth by the independent claims 16 and the dependent claims 17 to 20.

### BRIEF DESCRIPTION OF THE DRAWINGS

The invention, together with further objects and advantages thereof, may best be understood by referring to the following detailed description taken together with the accompanying drawings, in which:

FIG. 1a, 1b, 1c illustrate in principle a first embodiment of a pod in top and side views;

FIG. 2a, 2b, 2c illustrate in principle a second embodiment of a pod in top and side views;

FIG. 3a, 3b, 3c illustrate in principle a third embodiment of a pod in top and side views;

FIG. 4a, 4b, 4c illustrate in principle a fourth embodiment of a pod in top and side views;

FIG. 5a, 5b, 5c illustrate in principle a fifth embodiment of a pod in top and side views;

FIG. 6 illustrates in principle a sixth embodiment of a pod loaded with a dose before sealing of the pod;

FIG. 7 illustrates in principle a seventh embodiment of a pod loaded with a dose before sealing of the pod;

FIG. 8 illustrates in principle a eighth embodiment of a pod loaded with a dose before sealing of the pod;

FIG. 9a, 9b, 9c illustrates in perspective, top and side views a first embodiment of a sealed pod containing a dose;

FIG. 10a, 10b, 10c illustrates in top views embodiments of several similar pods containing differently sized doses;

FIG. 11a, 11b illustrates in top views embodiments of differently sized but similar pods containing differently sized doses;

FIG. 12a, 12b illustrates in top and side views an embodiment of two small pods containing separate doses, adapted for insertion together into a DPI;

FIG. 13a, 13b illustrates in side views a pod in relative motion to an embodiment comprising an opener and a suction nozzle;

FIG. 14a, 14b illustrates in side views a pod in relative motion to another embodiment comprising an opener and a suction nozzle;

FIG. 15a, 15b illustrates in side views a pod in relative motion to yet another embodiment comprising an opener and a suction nozzle;

FIG. 16 illustrates in a timing diagram a typical inhalation and pod motion according to the present invention;

FIG. 17 illustrates an embodiment of a dose carrier capable of carrying multiple pods, and

FIG. 18 illustrates a method of administering doses from pods.

### DESCRIPTION OF THE INVENTION

The present invention discloses a novel type of dose container, a so called pod, and its use as an enclosure optionally presenting a high barrier seal towards foreign matter, especially moisture, for a metered dose of a dry powder medicament intended for inhalation. The dose containers, pods, according to the present invention are available in different sizes and shapes to suit a selected dry powder inhaler device and a chosen dry powder medicament. Dose quantities may vary depending on the medicament and its potency, such that pods are available for filling with doses ranging from 50 μg to 50 mg and including dose masses within this range.

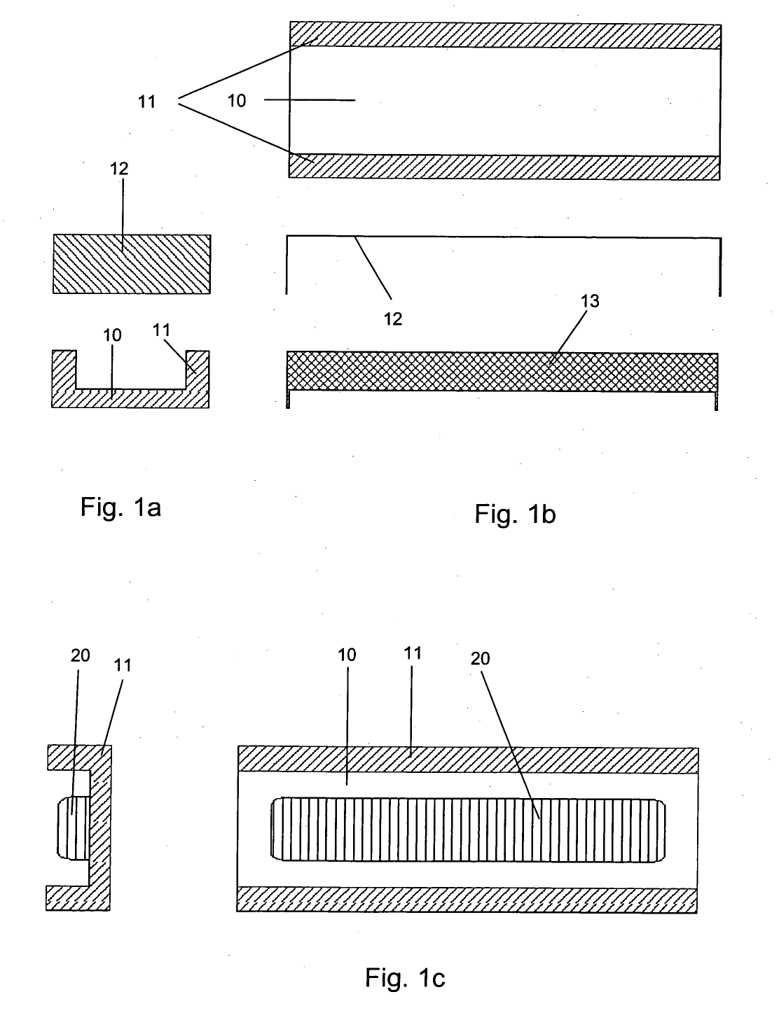

In FIGS. 1a, 1b, 1c, 2a, 2b, 2c, 3a, 3b, 3c, 4a, 4b, 4c, 5a, 5b, 5c, 6, 7, 8, 9a, 9b, 9c, 10a, 10b, 10c, 11a, 11b, 12a, 12b, 13a, 13b, 14a, 14b, 15a, 15b, 17, reference numbers 10-42 of the drawings like numbers indicate like elements throughout the three views of each of five different embodiments of pods suitable for doses of dry powder medicaments loaded onto a substrate of a pod as illustrated, presented here as non-limiting examples.

FIGS. 1a and 1b illustrate two side views and a top view of an embodiment of a pod 13 comprising a substrate 10, sealing surface 11 and a high barrier seal foil 12 with sharply angled end pieces. The seal is illustrated before it has been applied to the pod. FIG. 1c illustrates in top and side views a dose 20 loaded into the pod, ready for sealing.

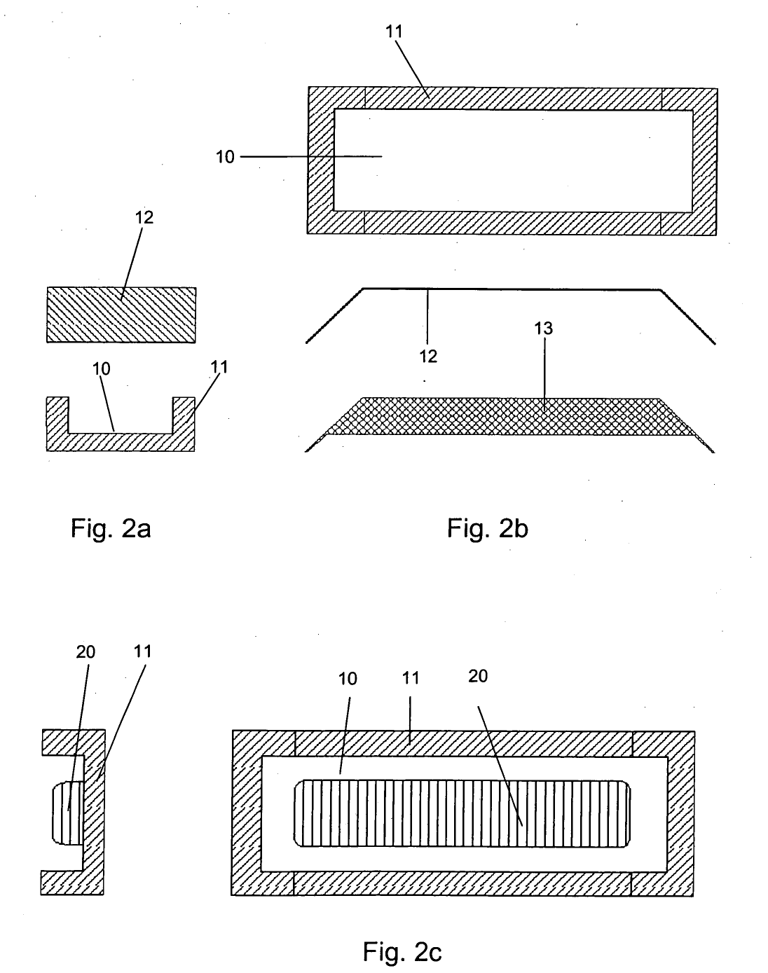

FIGS. 2a and 2b illustrate two side views and a top view of another embodiment of a pod 13 comprising a substrate 10, sealing surface 11 and a high barrier seal foil 12 with less inclined end pieces compared to FIG. 1b. The seal is illustrated before it has been applied to the pod. FIG. 2c illustrates in top and side views a dose 20 loaded into the pod, ready for sealing.

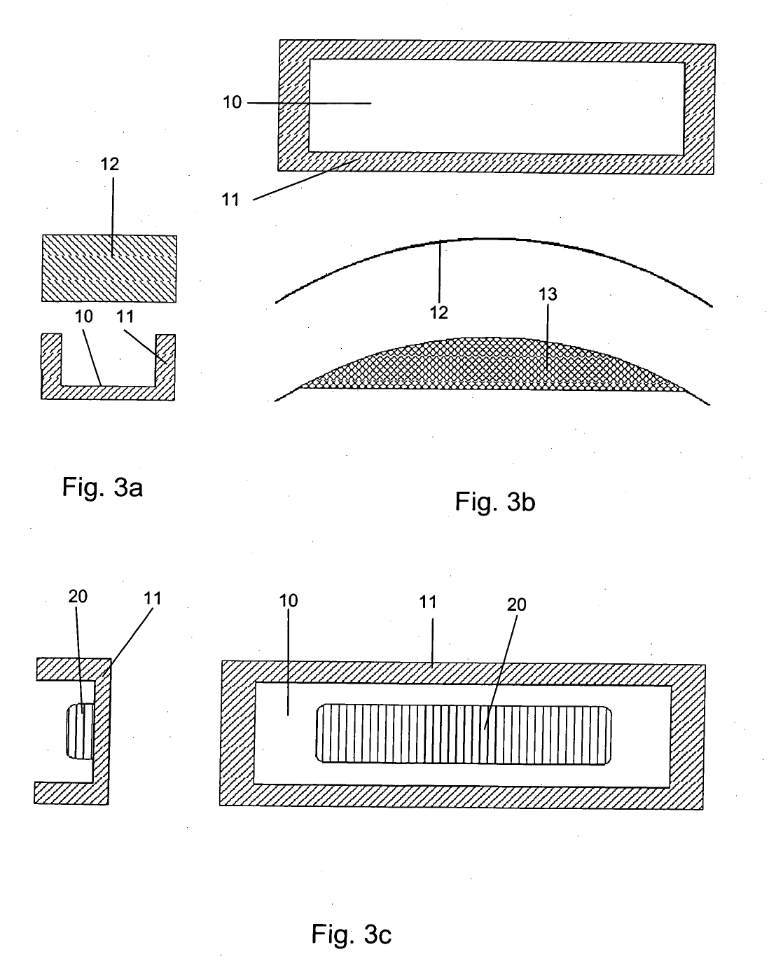

FIGS. 3a and 3b illustrate two side views and a top view of another embodiment of a pod 13 comprising a substrate 10, sealing surface 11 and a curved high barrier seal foil 12 with no sharp bends. The seal is illustrated before it has been applied to the pod. FIG. 3c illustrates in top and side views a dose 20 loaded into the pod, ready for sealing.

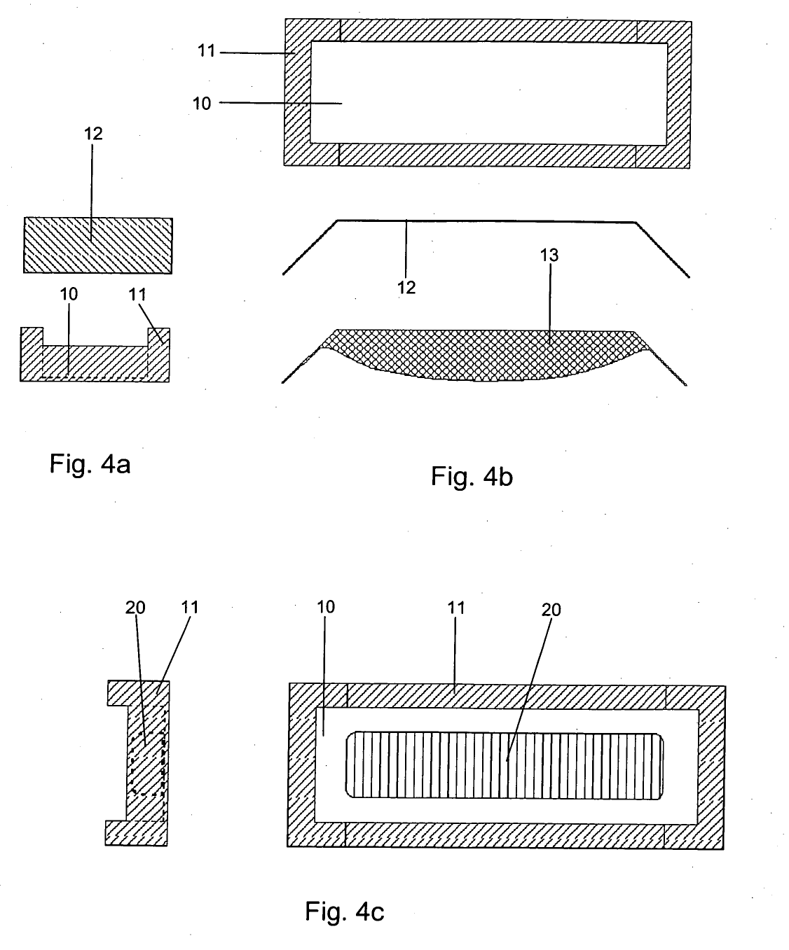

FIGS. 4a and 4b illustrate two side views and a top view of another embodiment of a pod 13 comprising a substrate 10, sealing surface 11 and a high barrier seal foil 12. The seal is illustrated before it has been applied to the pod. FIG. 4c illustrates in top and side views a dose 20 loaded into the pod, ready for sealing.

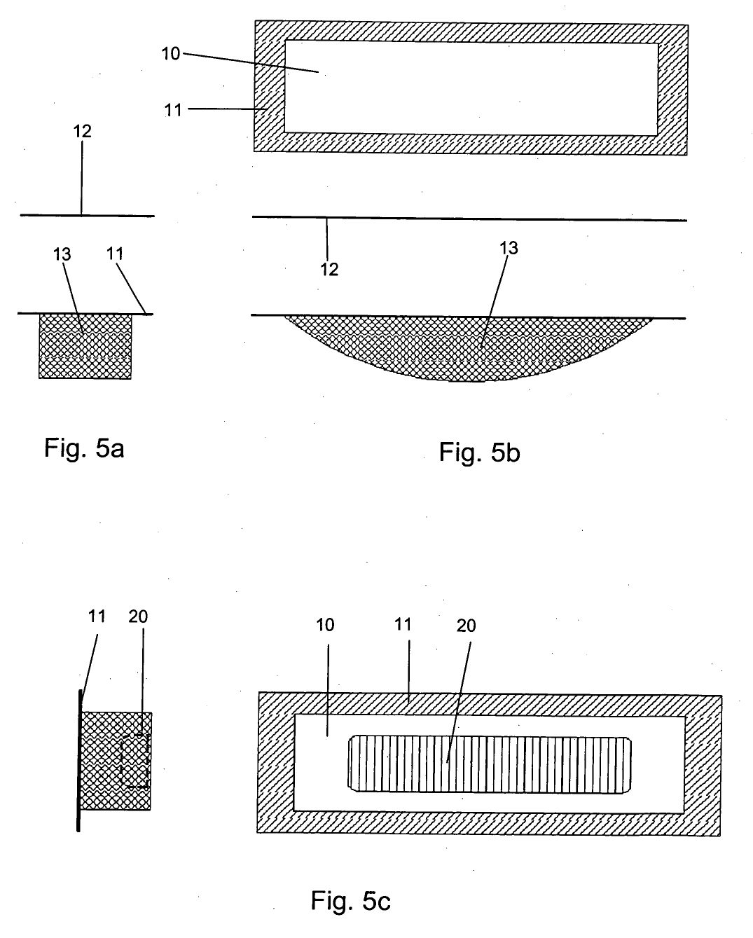

FIGS. 5a and 5b illustrate two side views and a top view of another embodiment of a pod 13 comprising a substrate 10, sealing surface 11 and a flat high barrier seal foil 12 with no sharp bends. The seal is illustrated before it has been applied to the pod. FIG. 5c illustrates in top and side views a dose 20 loaded into the pod, ready for sealing.

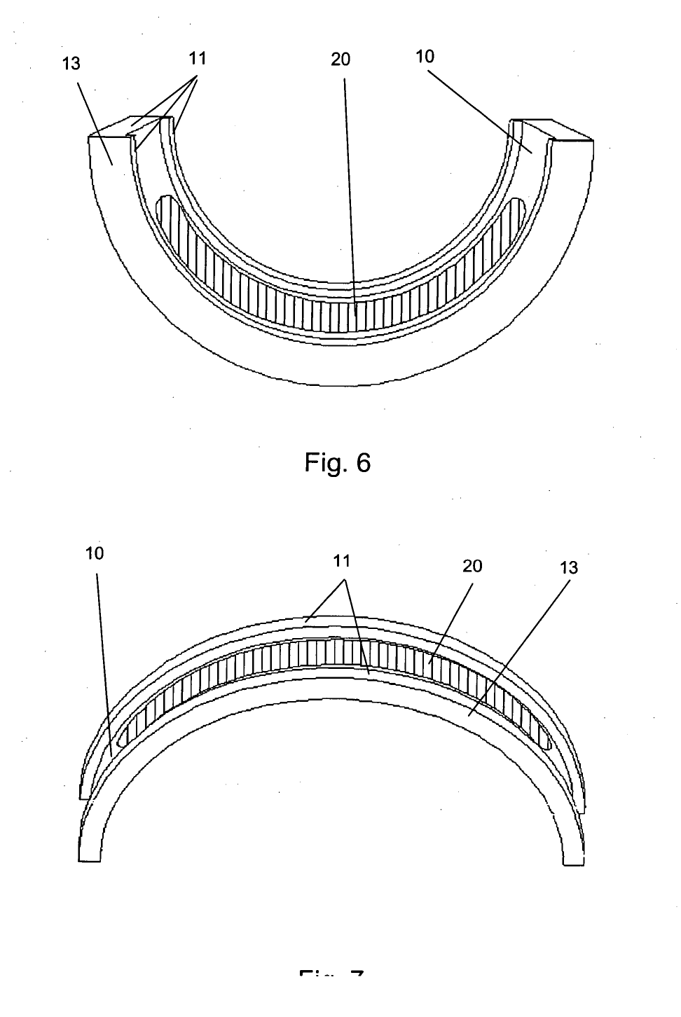

FIG. 6 illustrates an embodiment of a pod 13, using part of a cylinder, comprising a substrate 10 on an inside wall of the part cylinder, sealing surfaces 11 and a dose 20 loaded onto the substrate. The pod sealing foil is not illustrated.

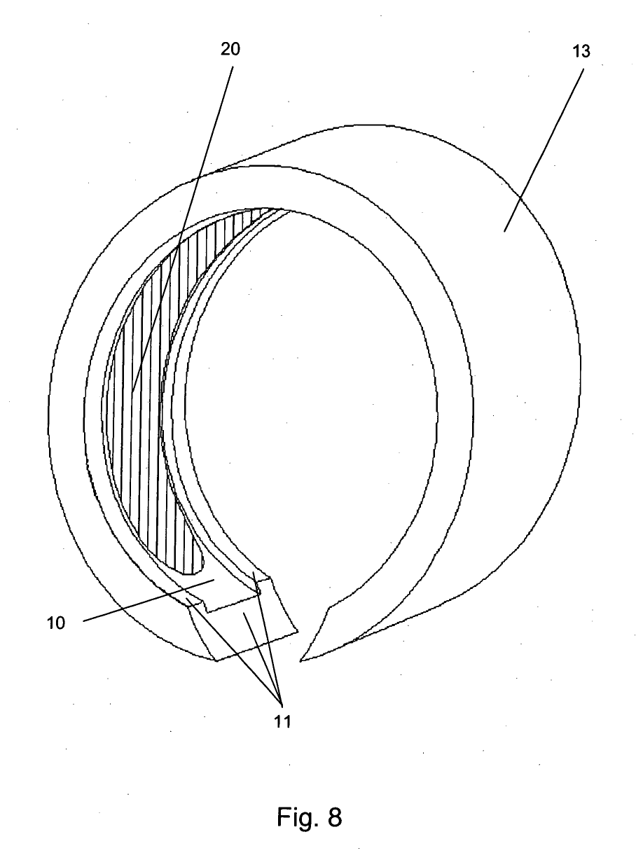

FIG. 7 illustrates an embodiment of a pod 13, using part of a cylinder, comprising a substrate 10 on an outside wall of the part cylinder, sealing surfaces 11 and a dose 20 loaded onto the substrate. The pod sealing foil is not illustrated.

FIG. 8 illustrates yet another embodiment of a pod 13, using almost all of a cylinder, comprising a substrate 10 on an inside wall of the part cylinder, sealing surfaces 11 and a dose 20 loaded onto the substrate. The pod sealing foil is not illustrated.

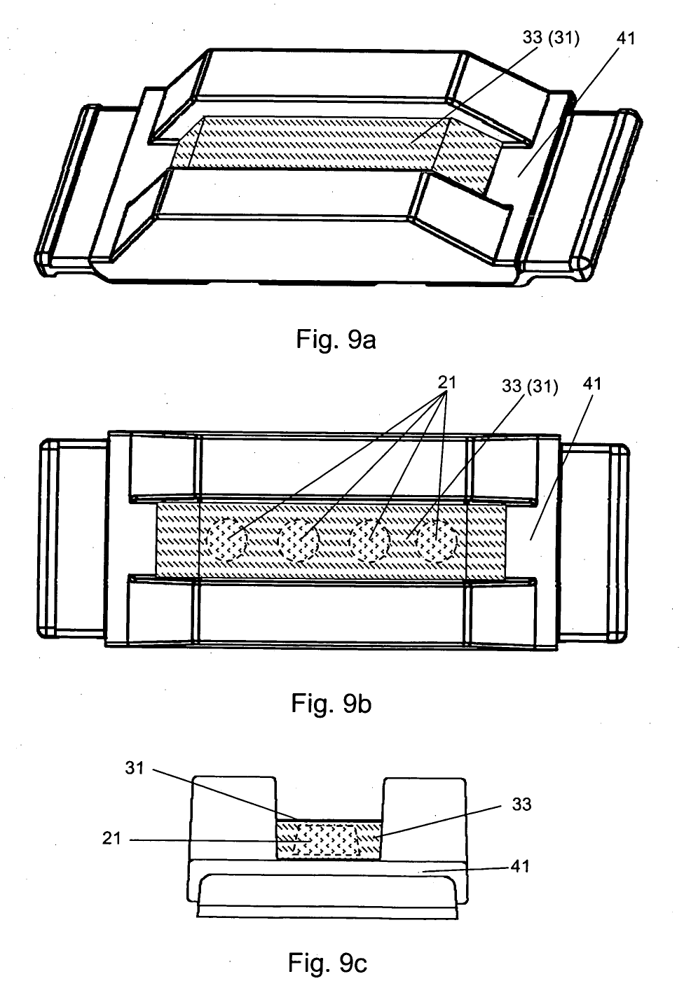

FIGS. 9a, 9b and 9c illustrating a carrier 41 carrying a sealed container 33 (seal 31) containing depositions 21 constituting a dose of a medical drug. The dose is hidden from view by the sealed pod, but nevertheless indicated in the illustration for the benefit of the reader.

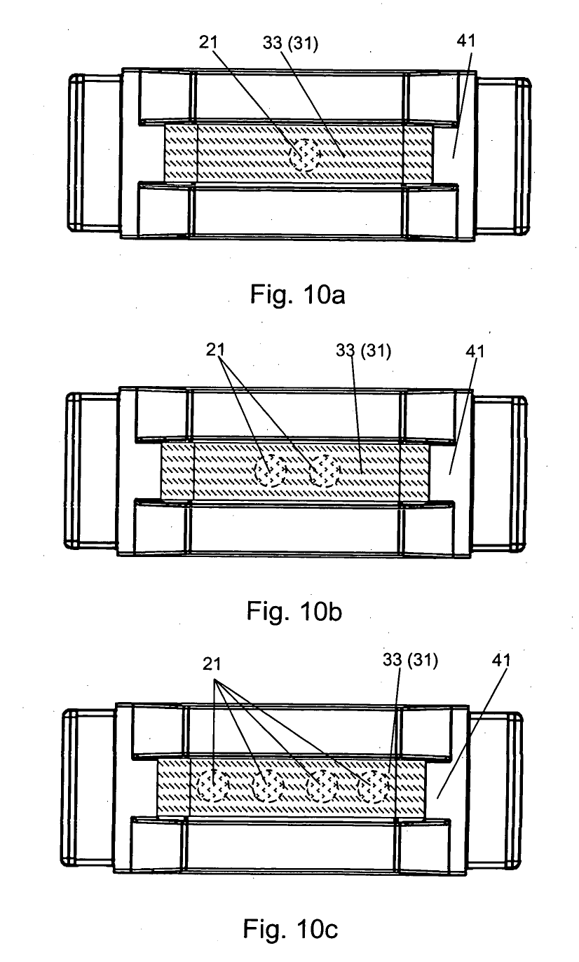

FIGS. 10a, 10b and 10c illustrating carriers 41 carrying sealed pods 33 (seal 31) containing different depositions 21 constituting differently sized doses of a medical drug. The doses are hidden from view by the sealed pod containers, but nevertheless indicated in the illustration for the benefit of the reader.

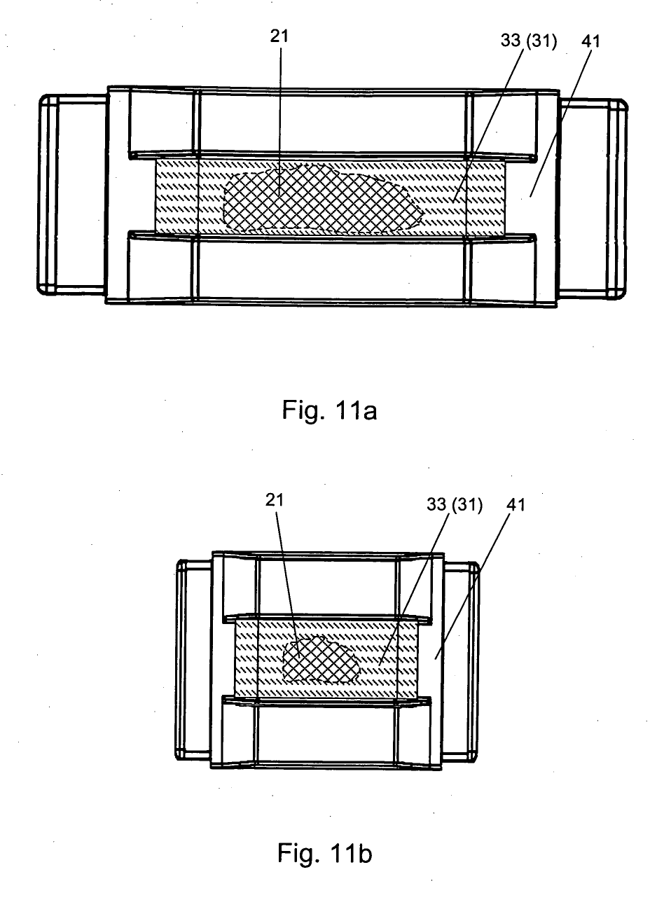

FIGS. 11a and 11b illustrating differently sized carriers 41 carrying differently sized sealed pod containers 33 (seal 31) containing different depositions 21 constituting differently sized doses of a medical drug. The doses are hidden from view by the sealed pods, but nevertheless indicated in the illustration for the benefit of the reader.

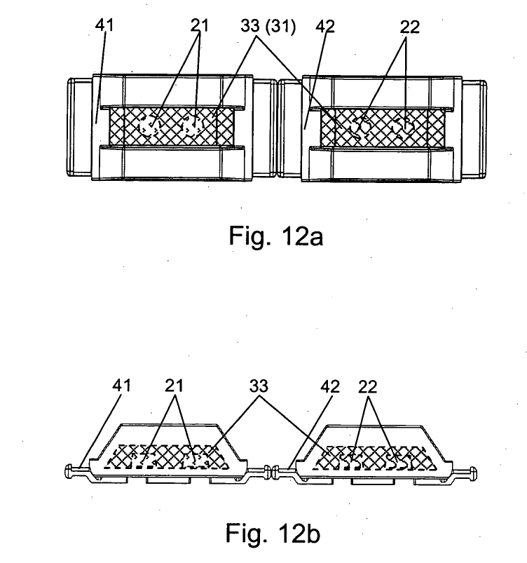

FIGS. 12a and 12b illustrating two carriers, 41 and 42, each carrying a sealed pod container 33 (seal 31) containing a dose 21 of a first drug and a dose 22 of a second drug respectively. The doses are hidden from view by the sealed pod, but nevertheless indicated in the illustration for the benefit of the reader.

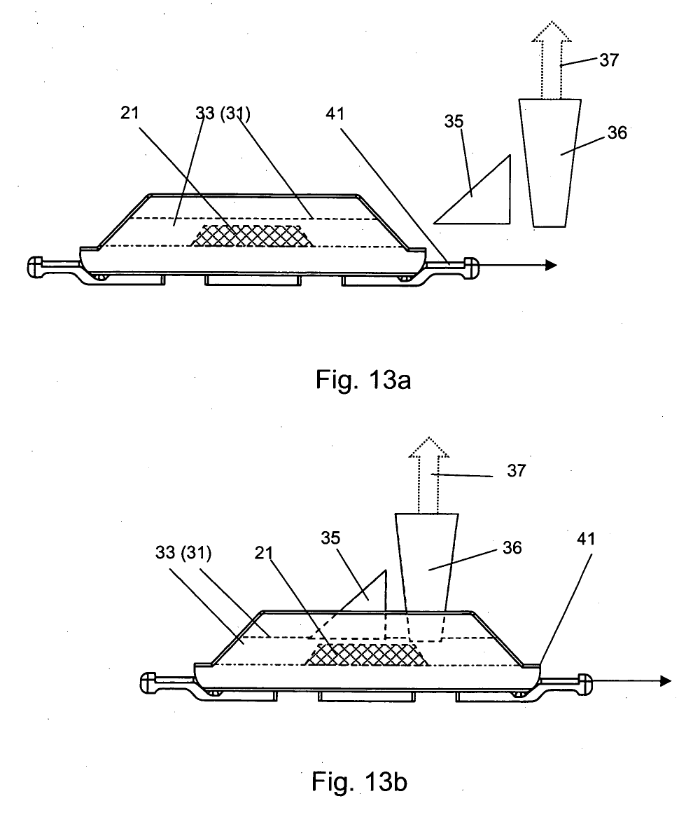

FIGS. 13a and 13b illustrating an embodiment of a moving pod carrier 41 carrying a pod 33 (seal 31) enclosing a dose 21, an opener 35 and a suction nozzle 36. The opener slits the sealing foil and the nozzle accesses the opened pod such that the dose may be released into the airstream 37 while the pod moves relative the opener and the nozzle.

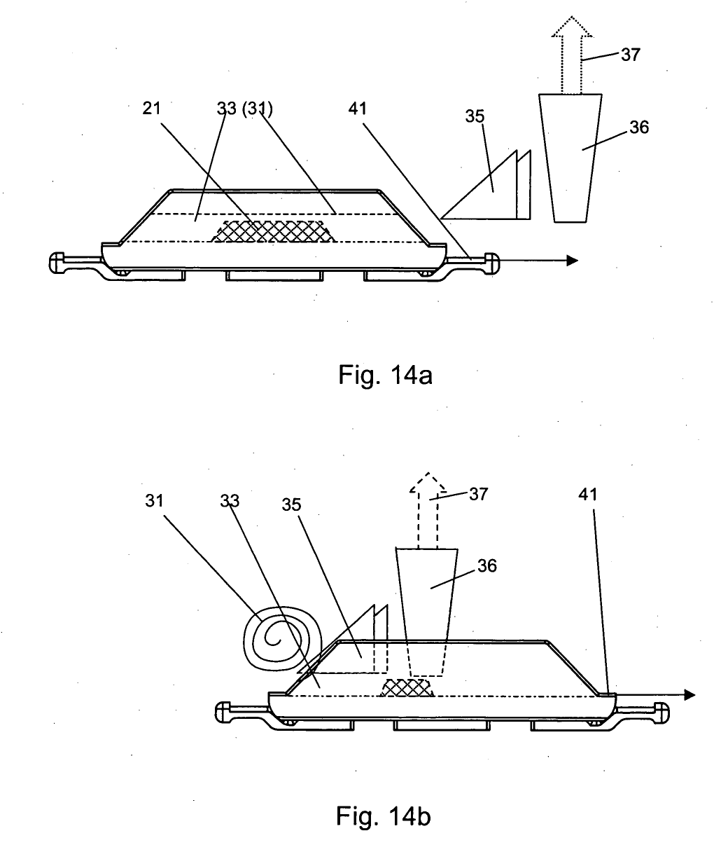

FIGS. 14a and 14b illustrating another embodiment of a moving pod carrier 41 carrying a pod 33 (seal 31) enclosing a dose 21, an opener 35 and a suction nozzle 36. The opener slits the sealing foil in two parallel slits and unfolds the opened sealing foil so that the nozzle gets open access to the opened pod such that the dose may be released into the airstream 37 while the pod moves relative the opener and the nozzle.

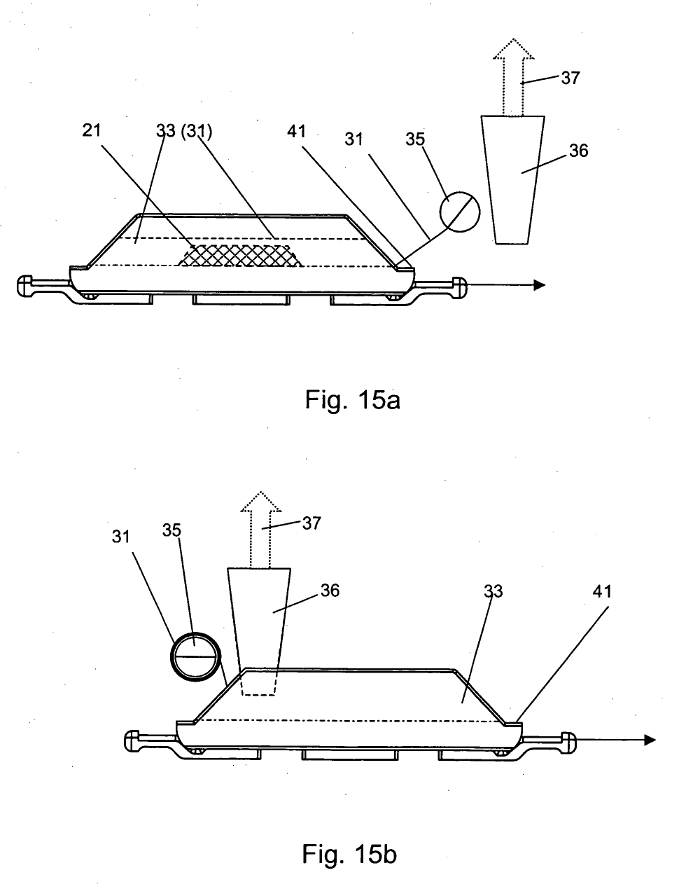

FIGS. 15a and 15b illustrating yet another embodiment of a moving pod carrier 41 carrying a pod 33 (seal 31) enclosing a dose 21, an opener 35 and a suction nozzle 36. The opener peels the sealing foil off from the pod container and unfolds the opened sealing foil so that the nozzle gets open access to the opened pod such that the dose may be released into the airstream 37 while the pod moves relative the opener and the nozzle.

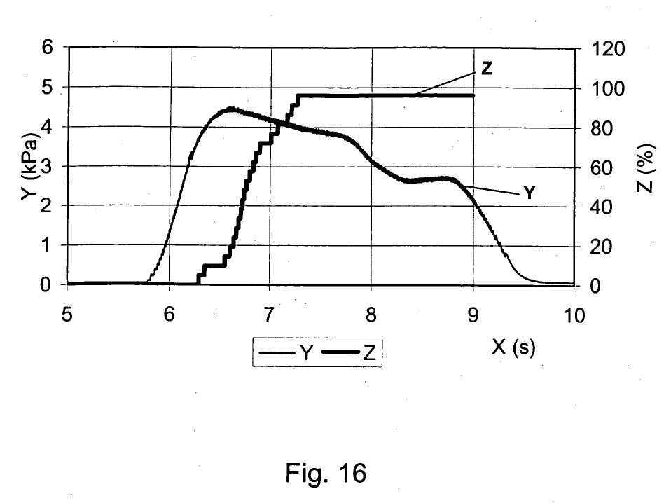

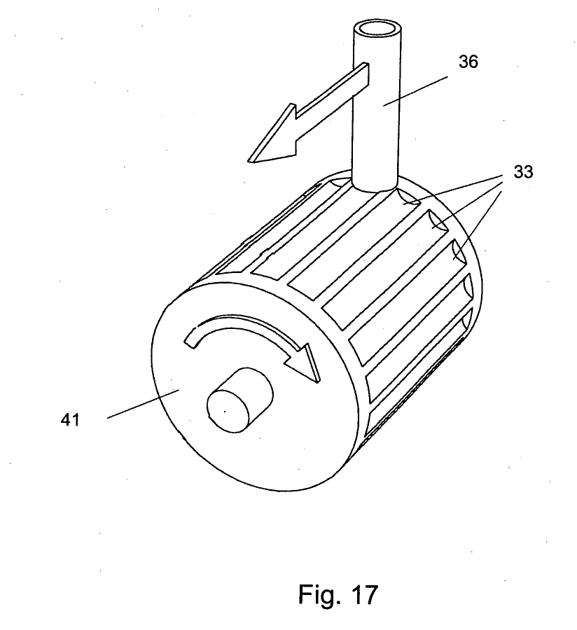

FIG. 16 illustrating a typical inhalation sequences carried out by a subject. Diagram curve Y represents the suction power in kPa provided by the subject over time X and curve Z represents pod motion from 0 to 100% in relation to a selected DPI. As can be seen, the motion does not begin until the suction is near the peak at about 4 to 5 kPa. The dose carried by the pod is thereby fully delivered before the suction power has dropped below 4 kPa.

FIG. 17 illustrating a pod carrier capable of carrying multiple pods. A pod is first selected and the pod carrier is then put in motion relative the suction nozzle or vice versa.

The illustrated embodiments are stylistic and to be understood as principal illustrations. For instance, the shapes of the pods, the substrates and the doses in the drawings are rectangular, but square shapes are of course possible, a square is a special case of a rectangle, in fact generally oblong or circular shapes are equally possible. Typical pod sizes range from 2×2 mm to 10×40 mm and dimensions within this range. Pod height varies between 0.5 to 5 mm.

A suitable pod size in terms of volume and physical dimensions depends on many factors, e.g. dose mass, dose porosity, type of powder formulation, preferred filling technique and the selected inhaler device.

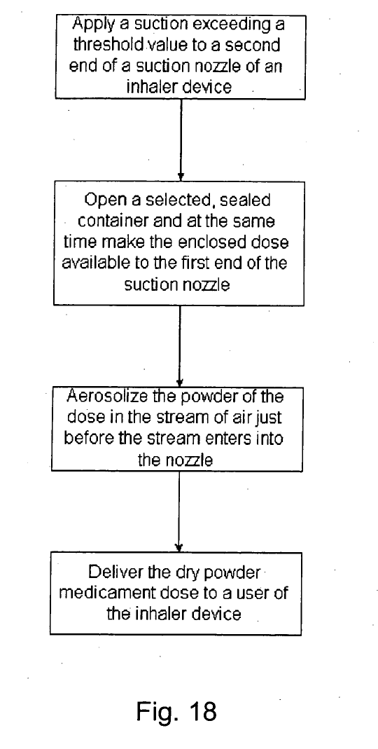

FIG. 18 illustrates a method of administering medicament doses from pods.

The main characteristic of a preferred embodiment of the present invention is that the sealed dose container, pod, is executed to make a simultaneous opening of the pod and release of an enclosed dose possible by the introduction of a relative motion between the pod and a suction nozzle. Preferably, in an adapted dry powder inhaler device, the pod is loaded in a movable slide, which is put in motion either inside the inhaler or such that the slide brings the pod into the inhaler device. The dry powder inhaler comprises means for moving the slide in or into the inhaler device, e.g. by manual force or electrical, mechanical, pneumatic or hydraulic energized members providing the moving means. Preferably, the suction nozzle is stationary inside the inhaler device. An opening element, an opener, is also present in the inhaler device. The opener is arranged to open the pod seal while the pod is in motion in or into the inhaler device and the suction nozzle is arranged to access the dose inside the opened pod as the motion continues. An inhalation is preferably applied to the suction nozzle before the relative motion of the pod begins. Preferably, for practical reasons, the opener and the nozzle are arranged as stationary components of the inhaler device and the pod is moved by the relative motion of the slide in or into the inhaler device. However, as a skilled person will realize, it is entirely possible to arrange a stationary pod and a relative motion performed by the opener and nozzle, which is within the scope of the present invention. In the context of the disclosure, use of words like “simultaneous” or “concurrent” refer to a relative motion of the pod relative the components opener and suction nozzle which are normally stationary in a dry powder inhaler, where the physical dimensions of pod, opener and nozzle are such that the relative motion brings the pod past the opener and nozzle, or vice versa, during a short time frame. An observer of the motion and the actions depending thereon would interpret the opening of the pod seal as “simultaneous” or “concurrent” with the access of the nozzle to the inside of the opened pod, even though, technically speaking, the seal must be broken before the nozzle can get access to the inside of the pod.

The timing of the motion is typically within a range from 0.1 to 5 s and preferably within a range from 0.2 to 2 s. The optimum time frame depends, inter alia, on the dose size, medicament formulation, type of inhaler device etc.

To protect the fine particle dose, FPD, up to the very point of aerosolizing of the dose a method of opening the dose container a fraction of a second before the dose starts to be aerosolized is described in WO 02/24266 A1 (U.S. Pat. No. 6,651,341), the relevant disclosure of which is incorporated herein by reference. In this context it is also important to prevent a voluntary or involuntary exhalation from a user of a DPI, who is about to inhale a dose, from reaching the selected dose, because of the high moisture content in the exhalation air. In U.S. Pat. No. 6,439,227 B1, the relevant disclosure of which is incorporated herein by reference, a device is disclosed, which closes the DPI, should the user exhale, so that exhalation air does not reach the dose container and the selected dose in the DPI. The device also controls the release of an opener and a suction nozzle such that the opener cannot open the container and inspiration air cannot begin to aerosolize the dose until a certain selected pressure drop is first present due to a suction effort by the user.

In a preferred embodiment of the present invention a dry, moisture-tight, sealed pod encloses a metered, directly loaded dose of a dry medication powder, where the pod and the enclosed dose are arranged in a DPI for dose delivery concurrent with an opening of the pod.

Another preferred embodiment of the present invention is a sealed pod having a flat or curved bottom acting as a substrate for a loaded, metered dose of a dry powder medicament, where a top sealing foil of the pod is arranged to be opened by an opener. For instance, the opener may penetrate and slit the foil from a first point of the pod to a second point of the pod. Thus, the first point of penetration of the foil is not necessarily the same as the second point. Another opener may peel the sealing foil off the top of the container, thereby making the dose accessible to a suction nozzle, while the relative motion is going on.

Another preferred embodiment of the present invention is a sealed pod, where the foil is arranged to be opened by the opener, which also unfolds the foil away from an enclosed dose; such that a suction nozzle may access and suck up the powder in the dose as the pod carrying the dose gradually becomes available to the nozzle because of the relative motion of pod vs. nozzle.

In a further preferred embodiment of the present invention, a selected, sealed pod is opened and the enclosed, metered dose is sucked up by a suction nozzle during a single inhalation, whereby the delivered fine particle dose by weight amounts to at least 30%, preferably at least 50% and most preferably at least 70% or more of the pharmaceutically active ingredient(s) of the metered dose.

In a further preferred embodiment of the present invention, more than one selected, sealed pods are opened in a defined sequence in a single motion and the doses enclosed in the respective pods are sucked up sequentially by a suction nozzle during a single inhalation.

Another preferred embodiment of the present invention is a sealed pod, inserted into a DPI, having a sealing foil arranged to be opened and unfolded away from an enclosed dose while in a relative motion into the DPI. Opening of the pod is triggered when at least a chosen threshold of suction pressure has been applied to a suction nozzle of the DPI, whereupon the enclosed dose is accessed by the suction nozzle when an air speed of sufficient turbulence has been developed into the nozzle to ensure efficient release and entrainment into the airstream of the powder in the dose.

Another preferred embodiment of the present invention is a sealed pod, which, when it is being opened and powder is being sucked up by the nozzle, presents consistent, even, symmetrical airflow conditions and equal dose accessibility for the airflow into the nozzle inlet from the beginning to the end of the pod motion, securing high quality dose delivery.

Another preferred embodiment of the present invention is a sealed pod, such that when the sealing foil is being opened and unfolded away from the dose, the dose becomes efficiently aerosolized into a suction nozzle provided suction has been applied to the nozzle, whereby retention of powder in the pod is minimized and not exceeding 30%, preferably not exceeding 20% and most preferably not exceeding 10% of the pharmaceutically active ingredient(s) of the metered dose by mass.

The pods according to the present invention are intended for insertion into a dry powder inhaler device, which arranges the pods, if more than one, for a user initiated administration and delivery of one or more metered dose per inhalation. In one embodiment of the invention one pod at a time is arranged by the inhaler for delivery of the enclosed, metered dose in a single inhalation by a user. The design of the inhaler controls how pods are to be inserted into the inhaler and the number of pods, which may be inserted and used before it becomes necessary to provide a new round of pods. Another embodiment requires that at least one pod is first mounted onto a pod carrier, which is then loaded into the inhaler.

As used herein, the phrases “selected from the group consisting of,” “chosen from,” and the like include mixtures of the specified materials.

All references, patents, applications, tests, standards, documents, publications, brochures, texts, articles, instructions, etc. mentioned herein are incorporated herein by reference. Where a numerical limit or range is stated, the endpoints are included. Also, all values and sub-ranges within a numerical limit or range are specifically included as if explicitly written out.

Preferably, the invention container, pod, uses dry, high barrier seals highly impervious to moisture and other foreign matter and is adapted for insertion into a dry powder inhaler device or the container may be adapted to be a part of an inhaler device.

“Dry” as used herein means that the, e.g., walls of the container are constructed from selected materials and/or materials treated such that the walls, especially the inside wall surface of the container, cannot release water that may affect the medication powder in the dose such that the FPD is reduced. As a logical consequence, container construction and materials should not be in need of processes suggested in the German publication DE 101 26 924 A 1 (US2003070679). As an example, gelatin is not a dry material and even after a special drying process gelatin still contains water. Generally, “dry” means the medicament FPD is not affected by the concerned material.

“High barrier seal” means a dry packaging construction or material or combinations of materials. A high barrier seal represents a high barrier against moisture and other foreign matter, and the seal itself is ‘dry’, i.e. it cannot give off measurable amounts of water to the load of powder. A high barrier seal may for instance be made up of one or more layers of materials, i.e. technical polymers, aluminum or other metals, glass, silicon oxides etc that together constitute the high barrier seal. If the high barrier seal is a foil a 50 μm PCTFE/PVC pharmaceutical foil is a particularly useful high barrier foil. For longer in-use stability metal foils like aluminum foils from Alcan Singen is a preferred choice.

A “high barrier container” is a mechanical construction made to harbour and enclose a dose of e.g. tiotropium. The high barrier container is built using high barrier seals constituting the walls of the container. The term “pod” is used in this document to describe high barrier container, characterized by having a bottom suitable for receiving a metered dose of a dry powder, either by volumetric or electrodynamic filling methods, and further characterized in being sealed by a foil, which may be slit open by a opener such that the enclosed dose may be accessed by a suction nozzle.

“Directly loaded” means that the metered dose is loaded directly into the high barrier container, i.e. without first filling the dose into e.g. a gelatin capsule, and then enclosing one or more of the primary containers (capsules) in a secondary package made of a high barrier seal material.

The high barrier containers to be loaded with medicament doses are preferably made out of aluminum foils approved to be in direct contact with pharmaceutical products. Aluminum foils that work properly in these aspects generally are composed of technical polymers laminated with aluminum foil to give the foil the correct mechanical properties to avoid cracking of the aluminum during forming. An example of a suitable aluminum foil for pods is type 115-0085E from Alcan Packaging Lawson Mardon Singen GmbH. This base foil is a laminate of 45 μm aluminum foil, 25 μm oriented polyamide film (OPA) on the dull side of the aluminum foil and 30 μm rigid PVC film on the bright side of the aluminum foil. This laminated foil can be cold formed into the desired shape. Sealing of the formed containers is normally done by using a thinner cover foil of pure aluminum or laminated aluminum and polymer. An example of a suitable aluminum sealing foil for pods is type 113-0049E from Alcan Packaging Lawson Mardon Singen GmbH. This sealing foil is a laminate of 9 μm aluminum foil and 6 μm polyester film on the bright side of the aluminum foil and a heat-seal laquer on the dull side of the aluminum foil. The container and cover foils are then sealed together using at least one of several possible methods depending on what materials and what foil construction is to be used, for instance:
using a heat sealing lacquer, through pressure and heat;
using heat and pressure to fuse the materials together;
ultrasonic welding of the materials in contact.

Preferably, sealing surfaces of pod versus foil are approximately 2.5 mm wide to ensure a high quality, leak-free seal.

The sealed pod of the invention that is directly loaded with a formulation of a medicament comprises a flat or curved substrate, e.g. a formed cavity in aluminum foil or a molded cavity in a polymer material, using a high barrier seal foil against ingress of moisture and other foreign matter, e.g. of aluminum or a combination of aluminum and polymer materials. The sealed, dry, high barrier pod may form a part of an inhaler device or it may form a part of a separate item intended for insertion into an inhaler device for administration of doses. The sealed pod may e.g. have the following data, as a non-limiting example:
Container internal volume: 100 mm³
Effective diffusion area: 46 mm²
Diffusion constant: 0.044 g/m² for 24 hours at 23° C. and differential Rh=50%

Expressed in a different way, the diffusion of water into the pod is in this example at a rate of 20 g/m³ per 24 hours at 23° C. at a presumed driving difference in Rh of 50%. For example, tests have shown that a sealed high barrier pod of the size above holding a dose of tiotropium preferably would not have a water transmission rate of more than 20 g/m³ for 24 hours at 23° C. and differential Rh=50% conditions to be suitable for an in-use time of maximum 2 weeks. The results may be transposed into a set of demands put on a different type of container, e.g. a blister. A blister of similar size to the pod in the example can be made using a typical high quality material like 50 μm PCTFE/PVC, which just meets the diffusion constant of the pod (=0.118 g/m² when re-calculated to at 38° C. and 90% Rh). If a pod containing a dose of tiotropium is intended to be in use for longer periods than 2 weeks, then a more moisture tight pod must be used to protect the FPD.

An inhaler providing delivery of a dose during the course of a single inhalation from a high barrier seal container produced from aluminum foils constitutes a preferred embodiment of an inhaler for the delivery of a dry powder medicament formulation. An Air-razor method as described in U.S. 2003/0192539 A1 is advantageously applied in the inhaler to efficiently aerosolize the dose when delivered to the user.

The present invention solves many prior art problems. In a preferred embodiment of the present invention, when applied to a suitably designed DPI, a certain suction power must first be applied to a mouthpiece of the DPI, before e.g. a valve opens to let air into the appropriate air channel in the DPI and further into a suction nozzle connected to the mouthpiece. This ensures that a fairly high air speed begins to build up around the inlet aperture of the suction nozzle. A foil opening operation is released simultaneously with opening of the air valve, but there is an interval before the opener begins to open the sealing foil at one end of the pod. In a relative motion, opener vs. pod, the foil is gradually opened and simultaneously unfolded away from the dose. The suction nozzle accesses the opened pod as the pod continues its motion towards the nozzle. Preferably, before the suction nozzle reaches the dose particles inside the pod, the air speed into the inlet aperture of the nozzle has already accelerated to a high speed, sufficient to release the powder particles and optionally de-aggregate particle aggregates. Keeping the distance generally constant between the inlet aperture of the suction nozzle and the substrate, i.e. the pod bottom, ensures that the shearing power of the air stream going into the nozzle is evenly distributed. Thus, the suction power is used to its full potential, regardless of where the powder is located on the substrate of the pod, presuming that the dose is present either in a single spot on the substrate or distributed over the area covered by the nozzle motion relative the pod. Retention is minimized. The time period between exposing the dose to the atmosphere and delivering the dose to the airways of a user is clearly extremely short, normally only fractions of a second, ensuring that the dose is unaffected by the surrounding atmosphere, when inhaled.

The unfolded edges of the cut foil may be folded back in the original position by the DPI, which closes, at least partially, the pod so that any powder retained in the pod does not fall out into the mechanisms of the DPI or into an air channel, where the powder may affect the operation of the DPI or present a risk to the user.

The description above of the invention provides a manner and process of making and using it such that any person skilled in this art is enabled to make and use the same, this enablement being provided in particular for the subject matter of the appended claims, which make up a part of the original description and including a medicament container enclosing a dry powder medicament dose for use in a dry powder inhaler, characterized in that a first component of the dry powder medicament consists of a fine particle dose of at least one pharmacologically active ingredient; the container constitutes a dry, high barrier seal, whereby the high barrier seal of the container prevents ingress of foreign matter, especially moisture, thereby preserving the original fine particle fraction of the dry powder dose; and the dry powder medicament dose in the container is adapted for either volumetric or electric field dose forming methods.

Additional embodiments include where the dry, high barrier seal is selected among the following materials, optionally in combinations: metals, including aluminum foil, thermoplastics, glass, silicon, silicon oxides.

As used above, the phrases “selected from the group consisting of,” “chosen from,” and the like include mixtures of the specified materials.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out. Terms such as “contain(s)” and the like as used herein are open terms meaning ‘including at least’ unless otherwise specifically noted.

What has been said in the foregoing is by example only and many variations to the disclosed embodiments may be obvious to a person of ordinary skill in the art, without departing from the spirit and scope of the invention as defined in the appended claims.

1. A medicament container for carrying a metered, dry powder medication dose, wherein
the medicament container constitutes a pod having a bottom acting as a substrate adapted for receiving a metered medication dose, the container adapted such that the metered dose, when releasably loaded into the pod, is protected from foreign matter including moisture after a sealing of the pod with a sealing foil;
the pod is executed such that the pod is opened and the dose in the pod accessed and released by an airstream going into a suction nozzle of an inhaler device while the pod is engaged in a single motion in or into the inhaler device.

2. The container according to claim 1, wherein
the sealing foil constitutes a high barrier seal foil, thereby transforming the sealed pod into a high barrier container for the enclosed metered dose.

3. The container according to claim 1, wherein
the pod has a flat bottom acting as the substrate.

4. The container according to claim 1, wherein
the pod has a curved bottom acting as the substrate.

5. The container according to claim 1, wherein
the sealed pod is adapted for insertion directly into an inhaler device such that an enclosed dose is directly delivered from the pod by the inhaler device.

6. The container according to claim 1, wherein
at least one sealed pod is attached to a pod carrier, which is adapted for insertion into an inhaler device, such that an enclosed dose is directly delivered from a selected pod by the inhaler device.

7. The container according to claim 1, wherein
when the sealing foil has been opened, the now open pod presents symmetrical, consistent airflow conditions and constant dose accessibility for an airflow into a suction nozzle, which is gradually accessing the dose;

8. The container according to claim 7, wherein
when the dose has been delivered from the pod, the sealing foil may be folded back in an original position, thereby enclosing at least partially any retained powder in the pod.

9. The container according to claim 1, wherein
the enclosed, metered dose becomes sucked up by a suction nozzle during a single inhalation act, whereby retention of powder in the pod amounts by weight to less than 30%, preferably less than 20% and most preferably less than 10% of the metered pharmaceutically active ingredients of the dose.

10. A method of delivering a metered, dry powder medicament dose enclosed in a sealed container to a user of a dry powder inhaler device, comprising the steps of
applying a user suction to a second end of a suction nozzle such that an airflow arises into a first end of the suction nozzle;
introducing a relative motion between the container, a so-called pod, and the suction nozzle, whereby the sealed container is opened and the enclosed dose is made available to the first end of the nozzle during the relative motion;
releasing the powder of the now accessible dose by making the powder of the dose in the opened up pod accessible to the stream of air just before said stream enters into the nozzle.

11. The method according to claim 10, comprising the further step of
arranging the pod, the dose and the nozzle motion relative the dose such that the air speed is evenly distributed around the inlet into the nozzle from a beginning to an end of the nozzle motion adjacent to the pod bottom, i.e. substrate, whereby good aerosolization conditions are maintained during delivery of the dose.

12. A dry powder inhaler device, comprising an opener and a suction nozzle, having an inlet and an outlet, said outlet in fluid connection with a mouthpiece, wherein
the inhaler device comprises a movable slide and means for moving the slide, said slide adapted for receiving at least one replaceable medicament container, pod, according to claim 1;
opening of a selected pod by the opener is commenced when a suction force is applied to the mouthpiece of the inhaler device and the movable slide carrying the pod is then put into motion to bring the pod past the opener and the inlet of the suction nozzle;
releasing during the relative motion the powder in the enclosed dose of the pod and entraining particles of said dose into an airstream going into the suction nozzle inlet.

13. The dry powder inhaler device according to claim 12, wherein
the pod is stationary in the inhaler device and the opener and the suction nozzle are moved by the slide relative the pod in order to bring the opener and the inlet of the suction nozzle past the pod.

14. The dry powder inhaler device according to claim 12, wherein
the opener unfolds the sealing foil, protecting the dose, away from the dose instantly after cutting the foil open, thereby making the dose accessible to a stream of inhalation air.

15. The dry powder inhaler device according to claim 13, wherein
the opener unfolds the sealing foil, protecting the dose, away from the dose instantly after cutting the foil open, thereby making the dose accessible to a stream of inhalation air.

16. The dry powder inhaler device according to claim 12, wherein
the inhaler comprises a breath-actuation mechanism preventing a selected pod from moving relative the opener and the suction nozzle or vice versa unless a suction force exceeding a preset level is applied to the mouthpiece of the inhaler device.

17. The dry powder inhaler device according to claim 12, wherein
the dose loaded on the substrate of the selected pod is released and entrained into a stream of inhalation air, resulting from the suction, concurrently with the pod sealing foil being slit open, whereby available dose powder in the pod is delivered by the inhaler device.

18. The dry powder inhaler device according to claim 12, wherein
the enclosed, metered dose in the pod becomes sucked up by a suction nozzle during a single inhalation, whereby the delivered fine particle dose amounts by weight to at least 30%, preferably at least 50% and most preferably at least 70% of the pharmaceutically active ingredients of the metered dose.

19. The dry powder inhaler device according to claim 12, wherein
the pod and the enclosed dose are arranged for a prolonged dose delivery by the inhaler device supporting a prolonged dose delivery.

20. The dry powder inhaler device according to claim 12, wherein
the suction nozzle and the pod are arranged such that the enclosed dose is not accessed by the suction nozzle until an air speed of sufficient turbulence has been developed into the suction nozzle, thereby ensuring efficient release and optional de-aggregation of the powder in the dose.

21. The dry powder inhaler device according to claim 12, wherein
two or more pods are loaded into the slide such that doses from the two or more pods are delivered as needed during a single inhalation by the inhaler device, either sequentially or simultaneously according to a pre-determined operational mode.

22. The inhaler device according to claim 12, wherein
the opened pod presents symmetrical, consistent airflow conditions and constant dose accessibility for an airflow into the suction nozzle, which gradually sucks up and aerosolizes the dose in the course of an inhalation;

23. The inhaler device according to claim 12, wherein
after the dose has been delivered from the pod, the sealing foil is folded back in an original position, thereby enclosing at least partially any retained powder in the pod.

24. The inhaler device according to claim 12, wherein
the single motion of the slide is within a time frame of 0.1 to 5 seconds from start to finish.

25. The inhaler device according to claim 12, wherein
the single motion of the slide is within a time frame of 0.2 to 2 seconds from start to finish.

26. The container according to claim 1, wherein
the pod is adapted to accept a dose load ranging from 50 μg to 50 mg and including dose masses within this range.

27. The inhaler device according to claim 12, wherein
the inhaler is adapted to accept a pod carrying a dose load in a range from 50 μg to 50 mg including dose masses within this range.

28. The container according to claim 1, wherein
the single motion of the pod is within a time frame of 0.1 to 5 seconds from start to finish.

29. The container according to claim 1, wherein
the single motion of the pod is within a time frame of 0.2 to 2 seconds from start to finish.
